(19) United States
(12) Patent Application Publication
(10) Pub. No.: US 2013/0046242 A1
(43) Pub. Date: Feb. 21, 2013

(54) Infusion tubing support clip

(71) Applicants: Linda Harris, Westfield, NJ (US); Norward Harris, Westfield, NJ (US)

(72) Inventors: Linda Harris, Westfield, NJ (US); Norward Harris, Westfield, NJ (US)

(21) Appl. No.: 13/136,877

(22) Filed: Aug. 15, 2011

(51) Int. Cl. A61M 39/00

(52) U.S. Cl. 604/174

(57) ABSTRACT

One end of a length of fabric material is folded under and then over itself in forming a pair of loops, in one of which a ring is captured and through the other which an infusion tubing of a central venous catheter is captured, with the other end of the length of fabric material being coupled with a spring loaded clip securing the fabric material and infusion tubing to the clothing of a patient undergoing a medical infusion treatment.

### CROSS-REFERENCE TO RELATED APPLICATIONS

None

### STATEMENT REGARDING FEDERALLY SPONSORED RESEARCH OR DEVELOPMENT

Research and development of this invention and Application have not been federally sponsored, and no rights are given under any Federal program.

### REFERENCE TO A MICROFICHE APPENDIX

Not Applicable

### BACKGROUND OF THE INVENTION

1. Field of the Invention

This invention relates to photopheresis treatments, in general, and to the administration of photopheresis procedures, in particular.

2. Description of the Related Art

Photopheresis or extracorporeal photochemotherapy is a treatment that utilizes ultraviolet (UV) light to activate a methoxsalen medication that works in the blood to prevent diseased cells from reproducing themselves. Two diseases that have benefitted from the use of photopheresis are Cutaneous T-Cell Lymphoma and Chronic Graft Versus Host Disease. The procedure is being employed in the treatment of blood and bone morrow diseases, as well.

During the photopheresis treatment procedure, a needle is placed in a patient's vein and blood is drawn into the photopheresis machine where it is separated. The white blood cells are there collected for UV light exposure and the other blood components are returned to the patient. At the end of the treatment, the white blood cells that were exposed to the UV light are then returned to the patient. In essence, the photopheresis machine constitutes a cell separator in which blood taken from one lumen of a central venous catheter is processed in removing the lymphocytes, and then returning them and the rest of the blood through a different lumen in the catheter. The treated lymphocytes are reinfused to stimulate an immune process for fighting the development of the diseases. As is known, photopheresis may be employed alone, or in combination with other treatments.

Although photopheresis treatments are generally painless, and although each treatment may last anywhere from two to four hours, and although the frequency of treatments as is determined by the physician may vary depending upon the patient's condition and diagnosis, the treatment requires the patient to lay in bed or recline in a chair while the blood is being taken, processed, and reinfused. Experience has shown, however, that the normal experiences of just twisting and repositioning oneself while reclining does have a tendency to lessen the securement of the surgical tape used to stabilize the catheter within the body site. Oftentimes, such movement is sufficient to dislodge the tape supporting the involved tubing to the extent that the photopheresis machine would stop and have to be started once again. This requires attention by a nurse of the photopheresis treatment team—and is something that it would be desirous to avoid, especially if the dislodgement were to reoccur.

### OBJECTS OF THE PRESENT INVENTION

It is an object of the present invention, therefore, to provide a new and improved manner of providing a support for the infusion tubing of the photopheresis treatment machine process.

### SUMMARY OF THE INVENTION

As will become clear from the following description, a preferred embodiment of the infusion tubing support of the invention is in the nature of a ribbon having a plastic ring on one end, and a mini-clip with a surface aperture on its opposite end. In operation, the ribbon is looped around the tubing and stretched through the ring to secure to the patient's clothing by means of the mini-clip. A coil joins the clip's aperture to an adjacent one on the ribbon to allow differing angular orientations in attaching to the clothing.

### BRIEF DESCRIPTION OF THE DRAWINGS

These and other features of the invention will be more clearly understood from a consideration of the following description, taken in connection with the accompanying drawings, in which:

FIG. 1A and 1B are top and side views, respectively, of a infusion tubing support clip construction in accordance with the preferred embodiment of the invention;

FIG. 2 shows the functional positioning of the infusion tubing support ready for use; and

FIG. 3 shows the infusion tubing support clip as employed on a patient with a chest catheter.

### DETAILED DESCRIPTION OF THE INVENTION

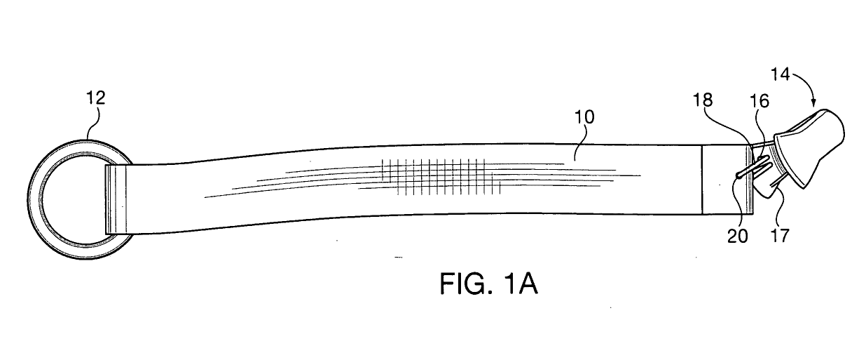

The infusion tubing support clip includes a ribbon strip 10 having a coupling means 12 (preferably in the nature of a ring) at one end, and a securement means 14 (preferably in the nature of a spring clip) at its opposite end. The coupling means ring 12 is of plastic composition having a one inch outer diameter while the securement means spring clip 14 is of a nature termed a “mini-clip”. The clip 14 includes an aperture 16 in one of its surfaces 17, 19, to receive a coil 18 in fastening through a comparable aperture 20 at the adjacent end of the ribbon strip 10. As FIG. 1B more clearly shows, the ribbon strip 10 is folded through, under and over the coupling means ring 12 and secured there in forming first and second loops 22, 24, while the coil 18 fits through the aperture 20 in the folded under end of the strip 10 at the securement means mini-clip. Primarily intended for use at hospital, long term care and nursing home facilities—although also available to be used where home infusion is to be had—, the ribbon strip 10 could be colored with the scheme of the facility, or could be decorated in any selected manner, as with cartoon characters for children's use in pediatric locations. Other dimensions for the coupling means ring 12 and securement means clip 14 may be employed, as well as different constructions for the “coupling” and “securement” components, as described below. In like manner, a variety of lengths may be chosen for the strip 10, depending upon where the infusion site is to be (for example, at an elbow, arm, shoulder or chest area of the patient), as well as the fabrication for which the strip 10 is to take. Utilization of the coil 18 between the aperture surface 17 of the mini-clip 14 and the aperture 20 in the strip 10 allows for a variety of angular orientations in selecting where on the clothing the clip is to secure.

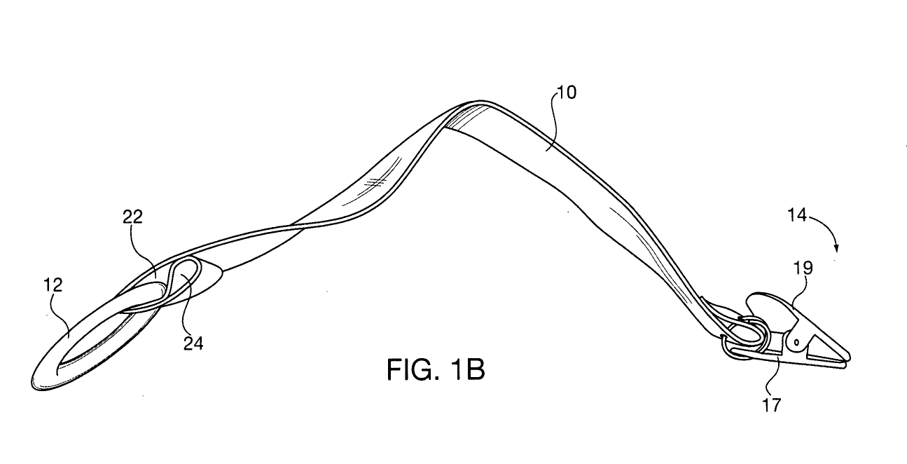

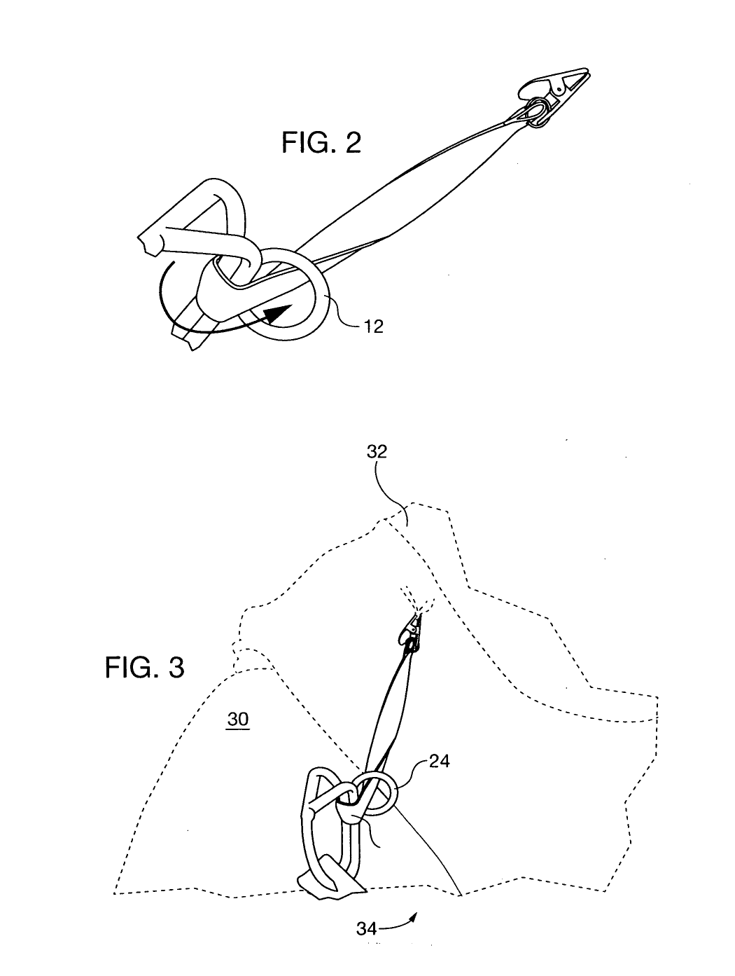

FIG. 2 illustrates the infusion tubing support arrangement shown in functional position, with the strip twisted over and fed through the coupling means ring 12, although the ribbon need not be so twisted over in actual use. In FIG. 3, the ring 12 is shown captured within the loop 22 and the infusion tube is shown captured within the loop 24. The mini-clip 14 is secured at a patient's garment, between the shoulder area 30 and the neck 32—the infusion site being indicated at 34.

In operation, investigation has corroborated that the infusion tube remains secure within the loop 24 as the patient undergoing treatment moves during the 2-4 hour interval of the photopheresis procedure, and the clip 14 remains secured to the garment. No need exists for a nurse to return to the site in repositioning any securement, nor to restart any machine that might otherwise shut down as a result of the securement coming loose. Depending upon manufacturing desires and costs, the entire infusion tubing support clip of the invention could be made disposable and could be adjusted to different parts of the clothing depending upon where the infusion site is located. With the strip 10 being of any length and of any material composition, all that becomes necessary is to select a length for the strip and a construction for the clip, in determining where the support is to be secured to the clothing. To such end, the coil 18 would then compensate for changes in orientation in having the clip remain in its initial placement.

A high practicality of use follows, minimizing the annoying side effects which characterize the prior art of having the procedure stop and start whenever the taped securement of the infusion tubing to the skin would become loosened to the extent of having to restart the machine procedure when stopping occurs. Such advantage will be appreciated in removing the emotional and psychological stress of the patient's having to sit or lie still in the chair or bed for several hours—which frequently becomes more problematic than the physical discomfort itself.

While the photopheresis treatments offer hope for the patient's recovery, the process of undergoing a long, arduous medical procedure is still not a pleasant one—and since there is no guaranty of its success, the additional emotional distress of having to lie still or only slightly moving which characterizes prior practice is preferably one to be avoided. The patient already feels quite isolated and helpless, in essentially being totally dependent on strangers for survival, no matter how competent they might be; adding to the discomfort is something to be minimized and done away with.

Such practicality of use of the invention will also be understood for a patient who is either allergic to the compositional make-up of the tape securement or who, in general, exhibits a measurable degree of skin sensitivity to its formulation. There, too, the infusion tubing support slip combination described above would offer a significant degree of benefits in administration.

While there has been described what is considered to be a preferred embodiment of the present invention, it will be readily apparent to those skilled in the art that modifications can be made without departing from the scope of the teachings herein. For at least such reason, therefore, resort should be had to the claims appended hereto for a true understanding of the scope of the invention.

I claim:

1. Infusion tubing support apparatus comprising:
a predetermined length of strip material;
first means at one end thereof for securing said strip material to an article of clothing worn by a patient undergoing a medical infusion treatment; and
second means at an opposite end of said strip material for coupling with a central venous catheter via an infusion tubing utilized during such treatment;
and with said strip material being folded at said opposite end to form a loop through which said infusion tubing passes in being captured there in place.

2. The apparatus of claim 1 wherein said second means includes a ring through which said strip material is passed leaving said loop external thereto to receive said infusion tubing.

3. The apparatus of claim 2 wherein said loop is formed by a folding of said first end of strip material under itself and secured thereat.

4. The apparatus of claim 3 wherein said ring is of a plastic fabrication of 1-inch diameter.

5. The apparatus of claim 1 wherein said first means includes a clip of opposing top and bottom spring loaded surfaces coupled with said strip material.

6. The apparatus of claim 5 wherein said first end of strip material is folded over onto itself and captured thereat in coupling with said clip.

7. The apparatus of claim 6 wherein one surface of said clip and said folded over first end of strip material are apertured, and wherein a coil is further included to pass through said apertures in coupling the clip to said first end of strip material.

8. The apparatus of claim 7 wherein said strip material is of the order of 6-inches in length.

9. The combination comprising:
a predetermined length of fabric material, one end of which is folded and secured under and then over itself in forming first and second loops;
a spring loaded mini-clip coupled by a coil to an opposite end of said fabric material;
a ring secured within said first fabric material loop; and
an infusion tubing of a central venous catheter
an infusion tubing of a central venous catheter secured within said second fabric material loop.

10. The combination of claim 8 wherein said ring is of a plastic fabrication of 1-inch diameter and wherein said fabric material is of the order of 6-inches in length.

11. The combination of claim 9 wherein each of a first surface of said clip and said folded-over end of said fabric material are apertured and wherein a coil is further included to pass through both said apertures in coupling the clip to said
